(19) United States
(12) Patent Application Publication
(10) Pub. No.: US 2023/0307101 A1
(43) Pub. Date: Sep. 28, 2023

(54) Distributed Multi-Device Information Dissemination System and Method

(71) Applicant: dacadoo ag, Zurich (CH)

(72) Inventor: Peter Ohnemus, Herrliberg (CH)

(21) Appl. No.: 18/189,887

(22) Filed: Mar. 24, 2023

Related U.S. Application Data
(60) Provisional application No. 63/323,253, filed on Mar. 24, 2022.

(51) Int. Cl. G16H 10/60; G06T 19/00; G06F 21/44; G10L 15/08

(52) U.S. Cl. CPC G16H 10/60; G06T 19/006; G06F 21/44; G10L 15/08

(57) ABSTRACT

Information associated with a person is securely collected, generated, and provisioned. At least one computing device secures at least some of received information (secured information) and stores the secured information on a distributed ledger system. A request to access the secured information is received. If the requesting device(s) do not have authorization to access the secured information, a virtual representation of the person with the request for authorization to access the secured information. The virtual representation of the person receives via the VR/AR environment from the person, the authorization, and the computing device(s) automatically provide, in response to receiving the authorization, the at least one other computing device with access to the secured information. Alternatively, where the at least one other computing device has authorization to access the secured information, the at least one computing device automatically provides the at least one other computing device with access to the secured information.

### CROSS-REFERENCE TO RELATED APPLICATIONS

This application is based on and claims priority to U.S. Provisional Patent Application Ser. No. 63/323,253, filed Mar. 24, 2022, which is incorporated by reference as if expressly set forth herein. This application further incorporates by reference the following commonly owned matters, as if each was set forth expressly in its respective entirely herein: U.S. patent application Ser. No. 15/313,513, now issued as U.S. Pat. No. 11,462,327, issued on Oct. 4, 2022; U.S. patent application Ser. No. 17/112,451; U.S. patent application Ser. No. 16/746,034; U.S. Pat. No. 8,996,314; U.S. patent application Ser. No. 15/778,999; U.S. patent application Ser. No. 16/536,158; and U.S. patent application Ser. No. 17/501,676.

### FIELD OF THE INVENTION

The present application relates, generally, to automated health data acquisition and, more particularly, to configuring each of a plurality of devices for providing health analysis, including as a function of virtual reality and augmented reality.

### BACKGROUND

Providing an assessment of an individual's health is complex, particularly in real-time as health-related events occur, and involves consideration of various factors. While some assess health in view of exposure to various hazards and health risks, other factors are often not considered, such as factors which derive from aspects of the individual's quality of life and lifestyle. Further, although broad definitions of health are known, such definitions are often qualitative, or semi-quantitative at most, and assessments often do not use a holistic approach for a quantitative determination of health.

Unfortunately, access to medical institutions, including hospitals, outpatient clinics, long term care facilities, laboratories, or the like, results in extremely high costs, often ranging in thousands of dollars for even the most routine of matters. Reducing costs associated with medical and health care remains a paramount concern.

Any holistic approach to measuring or assessing a person's health includes at least some degree of information based on self-assessment. Obtaining accurate and current self-assessed information can be problematic, particularly when participants do not provide information regularly and consistently. Unfortunately, participants often stop providing accurate and current self-assessed health-related information, particularly when platforms prompting for such information are not particularly easy, engaging, and/or attractive.

### BRIEF SUMMARY

In one or more implementations, a computer-implemented system and method are provided for securely collecting, generating, and provisioning information. At least one computing device receives, from each of a respective data collection sources, information associated with a person. The at least one computing device secures at least some of the received information at least as a function of at least one token to provide secured information. The secured information is stored on a distributed ledger system. A request to access the secured information is received by the at least one computing device from at least one other computing device. Thereafter, a determination is made whether at least one permission provided by the person gives authorization to the at least one other computing device to access the secured information. Where the at least one other computing device does not have authorization to access the secured information, the at least one computing device provides to the person, via a virtual reality and/or augmented reality (“VR/AR”) environment, a virtual representation of the person requesting authorization for the at least one other computing device to access the secured information. The virtual representation of the person receives via the VR/AR environment from the person, the authorization, and the at least one computing device automatically provides, in response to receiving the authorization, the at least one other computing device with access to the secured information. Alternatively, where the at least one other computing device has authorization to access the secured information, the at least one computing device automatically provides the at least one other computing device with access to the secured information.

In one or more implementations, automatically providing the at least one other computing device with access to the secured information further includes accessing, by the at least one computing device, the at least one token, and providing, to the at least one other computing device, the at least one token.

In one or more implementations, the at least one computing device provides via a VR/AR environment, the virtual representation of the person, wherein the request to access the at least some of the secured information is received by the virtual representation of the person.

In one or more implementations, the request to access the at least some of the secured information is received by the virtual representation of the person from a virtual representation of another person.

In one or more implementations, the at least some of the secured information was previously received from a plurality of respective sources. Moreover, automatically providing the at least one other computing device with access to the secured information further comprises accessing, by the at least one computing device, the information previously received from the plurality of respective sources; aggregating, by the at least one computing device, the information collected from each of a plurality of respective sources; and securing, by the at least one computing device, the aggregated information as a function of the at least one token.

In one or more implementations, the at least one computing device uses interactions between the person and virtual representation of the person via the VR/AR environment over time for machine learning and artificial intelligence.

In one or more implementations, the secured information includes information associated with the person's health. Moreover, the at least one computing device recognizes via the machine learning and artificial intelligence, at least one health condition associated with the user.

In one or more implementations, the secured information includes information associated with the person's health. Moreover, the at least one computing device monitors via the machine learning and artificial intelligence, aspects of the person's health.

In one or more implementations, aspects of the person's health include at least one of blood pressure, skin acidity, body temperature, heartrate, pupil movement, pupil shape, eyelid movement, iris information, sleep information, and stress.

In one or more implementations, the authorization received by the virtual representation of the person via the VR/AR environment is in spoken form from the person. The at least one computing device converts, using voice recognition technology, the spoken form of authorization to a digital form.

These and other aspects, features, and advantages can be appreciated from the accompanying description of certain implementations of the present disclosure and the accompanying drawing figures and claims.

### BRIEF DESCRIPTION OF THE DRAWINGS

Various features, aspects and advantages of the invention can be appreciated from the following detailed description and the accompanying drawing figures, in which:

FIG. 1 is a simple block diagram illustrating a plurality of devices that can be utilized and integrated in accordance with an example implementation of the present disclosure;

FIG. 2 shows example components and configurations of computing devices that can be used to implement the techniques described herein;

FIG. 3 is a block diagram illustrating a user or person, a virtual representation of a person, respective data sources, and interaction between a user or a person and a virtual representation of a person, in accordance with one or more implementations of the present disclosure;

FIG. 4 is a diagram illustrating an individual user, a respective virtual representation of the individual user, and a respective computing device associated therewith;

FIG. 5 is a diagram illustrating two respective virtual representations of individual users interfacing in connection with a network of a plurality of other virtual representations of people and individual users; and

FIG. 6 is a flow chart illustrating example processing steps, in accordance with an example implementation of the present disclosure.

### DETAILED DESCRIPTION OF THE EMBODIMENTS

By way of overview and introduction, the present application includes systems, techniques and interfaces for data processing, including for collection, design, modeling, simulating, and generating information associated with health of an individual. In one or more implementations, interactivity between a user and a virtual representation of a person (e.g., a virtual user or virtual other person) can be provided in various formats, such as in a virtual reality and/or augmented reality environment, holographically, or other suitable way. As used herein, the term “virtual reality and augmented reality environment” refers, generally, to input and output (“I/O”) provided by one or more computing devices, such as one or more display screens, smart glasses, VR goggles, hologram(s), camera(s), speaker(s), microphone(s), haptic device(s), or other I/O device(s), collectively, an “VR/AR environment,” which simulate a physical environment. Various kinds of input, including LIDAR or other imaging technology, can be provided substantially in real-time, thereby enabling for monitoring and measuring human action (and interaction), and one or more physical environments where a user is located. In such VR/AR environments, a virtual representation of a person interacts with a user. The VR/AR environment can be all virtual (e.g., virtual reality (“VR”), holographically, or other virtual implementation), all augmented reality (“AR”) or some combination thereof. The VR/AR environment can be provided via one or more apparatuses (referred to, generally, as a “VR/AR apparatus”), which can include a single device that is configured with multiple elements (display, audio input/output, etc.), or can include multiple devices, of which one or more are configured with multiple elements. Any respective aspect of a VR/AR apparatus, and use thereof, can depend on various factors, for example, and can include smart glasses, virtual reality goggles, computers, smartphones, holographic technology, or other interactive computing technology which can provide output to a user, measure user activity (or inactivity), and/or provide feedback.

The present disclosure includes technology that, in operation, can reduce the number of people populating medical institutions, including by enabling a user to use features shown and described herein at-home or in other convenient settings. One or more implementations of the present disclosure provide for augmented reality and virtual reality technology to operate in a new combined way which can preclude a need for utilizing institutional healthcare, including in times of emergency and crisis. A plurality of technology devices can operate in a holistic way to provide individual users with current and accurate information, including to increase a user's awareness and address any medical concerns in a timely way, which can avoid urgency. In some cases, users can be instructed via a VR/AR apparatus how to use one or more devices, such as for measuring, wearing something, using something, assembling something, or the like.

By providing a hybrid virtual reality and augmented reality environment, the present disclosure overcomes certain shortcomings associated with current virtual reality systems, such as QUEST face coverings by OCULUS and virtual reality headsets, which can be uncomfortable, especially over time as users have to keep their head straight or in particular orientation. The present disclosure includes technology to overcome such shortcomings, including by providing a synthesis of virtual reality and augmented reality system in which users can use smartphones, wearable devices, laptop computers, and other computing devices in conjunction with smart glasses and virtual reality goggles or other headwear for health-related monitoring, tracking, and prevention for digital health engagement and health risk quantification.

The present disclosure includes one or more sensor devices that can be configured to measure and track aspects of a person's health, such as by measuring blood pressure, acidity of skin, body temperature, heartrate, pupil movement, pupil shape, eyelid movement, iris information, sleep measurement, or the like. Some of the measured information can be used for monitoring health and behavioral characteristics of a user, and some can be used for security purposes, such as biometric security. In other implementations, stress can be determined as a function of measured and/or tracked aspects of the user health or activity. Information can be collected via one or more sensors that are provided in a single device, or a plurality of devices that operate in groups or individually. Such sensors and devices can be positioned at various locations of a person, such as head, arms, legs, feet, and torso. Body measurements from such sensors can be processed and used to calculate a health score, such as shown and described in commonly assigned U.S. patent Ser. No. 13/877,059, which is incorporated by reference in its entirety.

In one or more implementations, information can be derived from examining a user's eyes or parts thereof. Monitoring and measuring aspects and characteristics of the eye, such as the rate at which a pupil moves, a degree of pupil dilation, a direction a pupil is pointing, a degree and frequency of blinking or other eyelid and eye-related activity. Such measurements can produce information that can be processed as recognized as being symptomatic of one or more health conditions, such as stress, and can be used in the virtual reality and augmented reality environment of the present disclosure. For example, a virtual representation of a person can interact with a user, such as to alert the user of some intrinsic medical information or extrinsic activity information, or some indication of a medical or other type of condition, via a VR/AR apparatus. Further, the types of information collected can be used to calculate a user's composite value (e.g., a health score) or updates thereto, and the virtual representation of the person can interact with the user to provide the user's (or another user's) composite value.

In one or more implementations of the present disclosure, information collected from one or more respective sensors, information derived therefrom (e.g., a health score), or other information associated with a person can be stored on one or more secure distributed ledgers (e.g., on the blockchain). Health-related information can be distributed and accessed via a Web3.0 environment, including for secured user-based control and dissemination. Health-related information of an individual that is measured, collected, and generated in accordance with the teachings herein can be tokenized and stored securely on one or more distributed ledgers, including for revenue generation (e.g., licensing and sales), such as shown and described in commonly assigned U.S. patent application Ser. No. 15/778,999, which is incorporated by reference in its entirety. Moreover, respective health data points (e.g., cardio information, blood-glucose levels, blood pressure, pupil measurements, skin acidity, or virtually any health-related data point) can be stored in secured ledgers and controlled for use as a form of currency for revenue generation.

Further, the present disclosure integrates machine learning processes and artificial intelligence in an integrated synthesized virtual reality and augmented reality environment. For example, a virtual representation of a person (a “virtual person”) can be provided for a user, which operates as a form of a counterparty. In an onboarding phase, artificial intelligence can be used with a confidence interval to assess a person. For example, a person wears eyeglasses or other technology (e.g., a specialized earing, article of jewelry or clothing) that is equipped with sensors to generate certain health data points, as well as to interface with the user to receive information from the user. Thereafter, the data points and information received from the user can be processed, including to generate a composite value (e.g., a health score). Furthermore, over time artificial intelligence provides for an improved understanding of the user, including as extrinsic activity information, such as representing exercise (e.g., walking, running, bicycling, or other types of work-out activity), eating, sleeping, or other behavioral aspects, including those that indicate stress, can be provided via electronic monitoring and processing, and can be provided in a synthesized virtual reality and augmented virtual environment. Other environmental information can be measured and further provided via audio-visual output, such as representing surrounding noise levels, light (and darkness) levels, and air quality (e.g., pollen levels). Collectively, the data points can be provided in audio-visual output and configured as a virtual person who interacts with the user, such as a fitness or lifestyle coach, or as a virtual representation of the person him or herself, such as an avatar of the person, in various contexts.

In one or more implementations, the virtual representation of the person provided in a virtual reality and augmented reality environment is configured with access to information of the user or available to the user. For example, the virtual representation of the person can access the user's email, voicemail, calendar, documents, or other professional, personal, and confidential information of the user. In order to manage large volumes of email that inundate users, often on a daily basis, email messages can be accessed and prioritized, summarized, or otherwise provided by the virtual representation of the user in a virtual reality and augmented reality environment. Moreover, other kinds of correspondence and information can be categorized and, depending on particular contexts, information can be accessed automatically, and the virtual representation of the user can provide summaries of the information, portions of the information, or other aspect of the information to the user in a convenient and orderly way. Such information management provides reduces a user's stress levels, and also contributes to efficiency and improved work settings.

In addition, the virtual representation of a person disclosed herein can be configured to access health-related information of the user, including health data points, the user's current health score, patient histories, or other medical information. Such information can be processed and used to alert the user of a particular condition, as well as to initiate interactions and communication between a user and the virtual representation of the user. In one or more other contexts, approval from a user may be required before particular information is accessed, and, in such case, one or more messages can be sent from the virtual representation of the user to the user alerting him or her that attention to a particular item is needed.

One or more graphical user interfaces can be provided on a computing device operated by the user that provides for settings, permissions, customization, and tools for the user. For example, a user can select respective data sources which provides permission for the virtual person to access. For example, a user may want the virtual person to have access to health-related information (e.g., intrinsic medical information and extrinsic activity information), but restrict access to the user's email, documents, or other confidential information of the user. In such instance, the user can select graphical screen controls in a graphical user interface (e.g., sliders, checkboxes, drop-down lists, buttons, or the like) and define such access for the virtual person. As a particular context changes, such as the user changing his or her mind, the user may want to allow greater or lesser access to information for the virtual person. In such case, the user can make different selections in the graphical user interface, which can increase or decrease the kinds and amount of information available to the virtual person at a respective time. In one or more implementations, the interface can be physical, such as buttons, slider controls, or the like on a physical apparatus, such as a watch or other physical item.

Similarly, one or more controls can be provided via a graphical user interface for a user to define types of information and rules associated with disseminating the information to 3^{rd} parties, including other users (or other virtual representations of people). Certain information, such as a person's food and drink intake, including quantities may be permitted for dissemination by the virtual representation of the user. In other contexts, or as a default condition, other types of information may be retained in confidence and not disseminated without authorization from the user. For example, the virtual representation of the user may interact with a person other than the user, who requests information regarding the user's health. In such case, the virtual representation of the user can communicate with the user, such as to cause an email, SMS, or other transmission to be sent to the user, such as via a smart phone or other computing device, alerting him or her of the request.

Accordingly, the present disclosure supports secure and confidential interaction between the physical world and a virtual world, in which a virtual person interacts with a physical person (who may or may not be a person represented by the virtual person) in various ways and substantially in real time. The virtual person can operate in a coaching capacity and alert the user with particular health-related aspects (including the user's calculated health score and respective health data points), and with behavioral recommendations. For example, coaching can be provided automatically at various times. This can result in improvements in the user's health, as the user responds with exercise, improved eating, improved sleeping, proper use of medication, or other way to the virtual person in the mixed virtual reality and augmented reality environment. Such interaction can be particularly helpful, for example, for individuals suffering from memory loss or other ailments. For example, one or more sensors can monitor a person's movements, such as a user's gait, and which can be detected and processed for improved user interaction with the virtual person.

Various interactions via the virtual person assisting with the user's medical, personal, and professional affairs are thus provided herein, including by accessing kinds of information shown and described herein associated with the user. As a function of machine learning and artificial intelligence, a user's behavioral patterns can be discerned automatically, and information representing such patterns can be processed for improved and evolving understandings of the user and for improved interactions between the user and the virtual person. Moreover, physical and/or medical training can be provided, including a virtual training room or other location provided as a function of virtual reality and/or augmented reality. In one or more implementations, a baseline composite value (e.g., baseline health score) can be calculated, including by imputing one or more values for calculating the composite value that can be associated with the user. As additional information regarding the user becomes available, the user's health score, for example, can be updated.

In one or more implementations, the hybrid virtual reality and augmented reality environment can be provided via technology worn by a user, such as glasses, headphones, goggles, and/or holographic technology, of which at least some of which can be equipped with sensors and other technology. In addition, or in the alternative, display screens and external speakers can contribute to the hybrid virtual reality and augmented reality environment, without requiring a user-worn apparatus.

It is to be appreciated that the virtual person of the present disclosure can operate as a virtual twin of the user and can be provided with access to various information sources shown and described herein. In a virtual setting, such as a virtual conference call, one or more computing devices implementing artificial intelligence can enable the virtual “twin” to participate on behalf of (or in conjunction with) the user during the virtual conference call. Since, in certain implementations and usages, the virtual twin can have access to significant amounts of information of the user, a user can participate without actually physically participating. One or more computing devices can aggregate information associated with the user, which can be available for the virtual person (e.g., the virtual twin) substantially in real time during one or more virtual events. Depending on a particular implementation, to other participants in the setting, the virtual twin appears to be the user himself or herself. Moreover, as a virtual person participates, information can be provided to the user substantially in real time, such as in case the virtual person requires something from the user. For example, authentication information may be required of the user in a given context and the virtual person can alert the user accordingly.

Thus, the present disclosure provides for a new form of data security and privacy, in which the virtual representation of a person operates as a firewall of sorts. Users no longer have to view and work with data directly, but rather maintain a level of separation from sensitive data via the virtual representation of a person, which provides the information to the user. Further, the architecture of the present disclosure can be viewed as a form of dual authentication. For example, a user must provide sufficient authentication for access to, for example, a VR/AR apparatus for accessing a virtual reality and augmented reality environment.

In one or more instances, a virtual representation of a user can operate substantially independently in order to interact with a third party, which could be a physical person or a virtual representation of a different person or group of people. The virtual representation of the user may try to access information that requires dual authentication or other access security. In such case, the virtual representation of the user can make an authentication request to the user, such as over a different communication channel (e.g., vis SMS, email, social media, or the like), thereby providing a second authentical layer.

In another example, the virtual representation of a person can be configured to operate substantially independently in respective contexts, and information may be requested by a 3^{rd} party that requires authorization from the user. In such example the virtual representation of the user is not provided with permission or authentication to access the information independently. Continuing with this example, the virtual person can alert the user that information is being requested, such as via SMS, e-mail, telephone, or other medium. The user can then provide the information to the virtual person, such as via voice-recognition technology, optical character recognition, response in an interactive graphical user interface, or other suitable format. Thereafter, the virtual person can access the information and relay the information to a requesting party. For example, the virtual person may simply speak the information to a physical person via a remote communication session. In other contexts, the virtual person may transmit information via email, file transfer protocol, or other suitable way. In this way, the present disclosure can reduce errors prone to humans, such as to ensure a correct email attachment provided or the recipient email address is correct.

Thus, in one or more examples, the virtual person can be configured to operate in a coaching capacity, such as by monitoring health information associated with the user and encouraging the user in various ways. That could include assistance during an exercise regimen, assistance with dieting, reminders or monitoring of medication use, shopping assistance, or virtually any other capacity to assist a user with a task or chore. When operating in a coaching capacity, the virtual person periodically or continuously learns about the respective user, including by presenting a calculated health score, respective health data points, behavior of the user, or other information that may be used for improving the user's health. In addition, as the virtual person becomes increasingly familiar with the user, the virtual person provides advice and recommendations to assist the user at respective times and in particular contexts. For example, the virtual person may recommend that a user increase or decrease exercise, while the user's health is being assessed. In another context, the virtual person may recommend drinking water, taking medicine, or other activity that can benefit the user. In yet another example, a user is prone to diabetes. Continuing with this example, the user can wear smart glasses that include sensing technology, virtual reality and/or augmented reality display, and audio output (e.g., headphones or speakers). The virtual person, via the glasses, provides various kinds of health information, including the user's health score, one or more calculated health risks, and individual health data points. While acting in a coaching capacity, the virtual person can confer with the user substantially in real time.

In other contexts, the virtual person can be configured to operate in a representative capacity, including to participate in conferences, calls, or other interactive events. Such representation can be provided anonymously or not anonymously, depending upon the context and preferences of the user. Providing a virtual person in an anonymous representative capacity can provide a secure and confidential information source for users, for example, in connection with sensitive matters. Individual users seeking information on addiction, anxiety, mental health, or other topics that may have an associated stigma can engage others anonymously via the virtual person. Such users are more inclined to be honest and open to reveal information about themselves using the technology shown and described herein.

It is recognized that users' medical health information, professional information, and personal information are sensitive and people have significant expectations of privacy and security when dealing with such data. Further, the present disclosure addresses security and privacy concerns relating to major data warehouses (e.g., storing data from internet social networks, internet search engines, financial institutions, healthcare institutions, educational institutions, chat data, biometric databases, hospitality institutions (e.g., hotels, bed and breakfast housing, or the like), to name just a few, which have suffered significant data breaches in the past, collectively affecting billions of people. The architecture of the present disclosure provides improved security via one aspect regarding storage and transmission of data, including on a distributed ledger, and via an access layer in the form of a virtual person provided in a virtual reality/augmented reality environment. Biometric security, including as a function of sensors monitoring the user, can be used to unlock or restart disabled smart glasses or other apparatus or to ensure the wearer is authorized (e.g., is the owner). Providing sensitive information by restricting access by a virtual person, in one or more implementations individual users are not provided with access to the information directly.

Accordingly, the computerized hardware and software architecture of the present disclosure provide improvements in data privacy protection and security are provided. Such improvements regard security and privacy protection of health data points and parameters contributing to calculation of a user's health score (e.g., sensitive medical data collected automatically by sensors worn or in contact with individuals, as well as information received and validated from medical professions). Further, security and privacy protection is provided for professional and personal documents such as word processing documents, PDF files, spreadsheets, database content, e-mail correspondence, SMS and other instant text messages, social network content, images, audio content, video content, web site content and information. At least some of such information can be stored via as distributed ledgers (e.g., on the blockchain) and secured via, for example, public/private key cryptography, software tokens, and/or hashing, which further ensures data privacy and security.

Moreover, the architecture of the present disclosure which can, in one aspect, store, transmit encrypt, tokenize, and/or hash data including via distributed ledgers, provides a second layer of security and privacy by restricting access to information via a virtual representation of a person in a virtual reality and/or augmented reality environment. The present disclosure improves upon security, including via a multi-phased approach that includes generating and accessing information of a user, processing the information for secure storage and use, and providing to a user the information via a virtual reality and augmented reality physical environment. Such physical environment can include, for example, one or more wearable devices that generate, provide (e.g., by display audio, haptic, or other suitable output), and/or transmit respective measured or accessed intrinsic medical information and or extrinsic activity information.

In one or more implementations, dual authentication is used at various stages or times during use of a virtual reality and augmented reality apparatus. Further, use of virtual reality and augmented reality technology prevents certain data breach risks, such as relating to keylogger malware, man in the middle attacks, or other types of security and privacy risks.

The present disclosure deploys machine learning and artificial intelligence vis-á-vis measured intrinsic medical information and extrinsic activity information to generate a form of a health IQ, comprised of a distributed ledger, populated via devices configured with hardware for sensing users and generating and transmitting data to one or more devices. At least some of the data are provided on distributed ledgers, which can be accessed by a virtual person in a virtual reality and augmented reality environment, thereby providing for communication with the user. The resulting whole is far greater than the sum of the respective parts.

In one or more implementations, when operating in a coaching capacity, the represented person in a virtual reality and augmented reality environment can effectively be an exercise machine for a user, including for aerobic exercise, strength training (e.g., resistance training), cardio-vascular workouts, stretching, yoga, mobility exercises, and the like.

A benefit of using machine learning and artificial intelligence in accordance with the teachings herein is the ability for continual learning, recognition, and association of virtually countless aspects of a user. As a user continues to interact over time with the virtual representation of the user, and as the virtual representation of the user continues to interact with other users or other virtual representations of users, a tremendous library of data can be amassed that provides significant value to the user and other users. Such information that is gleaned over use is effectively owned by the user, which is available for the user to sell, license, barter, give away, or otherwise do as he or she wishes. Examples of users licensing and entering into smart contracts covering aspects of their health data is shown and described in commonly assigned U.S. patent application Ser. No. 15/778,999.

Various measures for making information available are envisioned herein. A user can license the virtual representation of the user to one or more other users. For example, the virtual representation of the user can be licensed by providing lectures or presentations of information or other kinds of information sharing in one or more virtual reality and augmented reality environments. The virtual representation of the user can meet with individuals in virtual settings for information sharing. In another example, information can be packaged and transmitted securely, such as over distributed ledgers, in response to commands generated and/or issued by the virtual representation of the person. Thus, the information that is generated in accordance with the teachings herein can be licensed or sold to others.

Thus, a user can experience, with or without the virtual representation of the user, other people's virtual representations in interactive presentations, conferences, discussions, or the like. Alternatively, or in addition, information from other users can simply be amassed, including via distributed ledgers, including by being available to the virtual representation of the user and used for further interaction and education provisioning with the virtual representation of the user. The present disclosure provides for a new form of crowd-sourced information and dissemination, including by providing a marketplace of continually evolving data processing and access.

In one or more implementations, one or more uniform, non-proprietary file formats is supported that can provide various kinds of health and activity information. This increases the availability of many kinds of information, including health information, to the virtual representation of the user, which can access such information as a function of the common file format(s).

As noted here in, the present disclosure supports measuring acidity of a person's skin, for example, for measurements and determination associated with glycoses. Such analysis can contribute for diagnostics of one or more medical conditions, such as diabetes. While not a clinical tool, per se, that identifies and quantifies risk factors as causes of overall health risk, the present application operates to derive a plurality of measurements. The measurements are combinable or otherwise used to calculate a relative measurement representing a current state of health of an individual, as well as the probable best avenues for the improvement of state of health. The presence or absence of any particular measurable factor in an individual that is found to be quantitatively and significantly correlated with health risk, independently of the direction of probable causality, is paramount to determining a representation of a current state.

The present disclosure supports a form of a health metaverse, which supports a form of a cryptowallet and knowledge gathering and sharing. Improved at-home healthcare is provided, which can include virtual reality and augmented reality. An immersive environment of a hybrid world (an augmented world) and a virtual world (such as with brain training) is supported for information generation and sharing. In one or more implementations, information can be provided anonymously in a crowd-sourced database, wherein information is bundled with other user's information without providing a direct connection to any one individual.

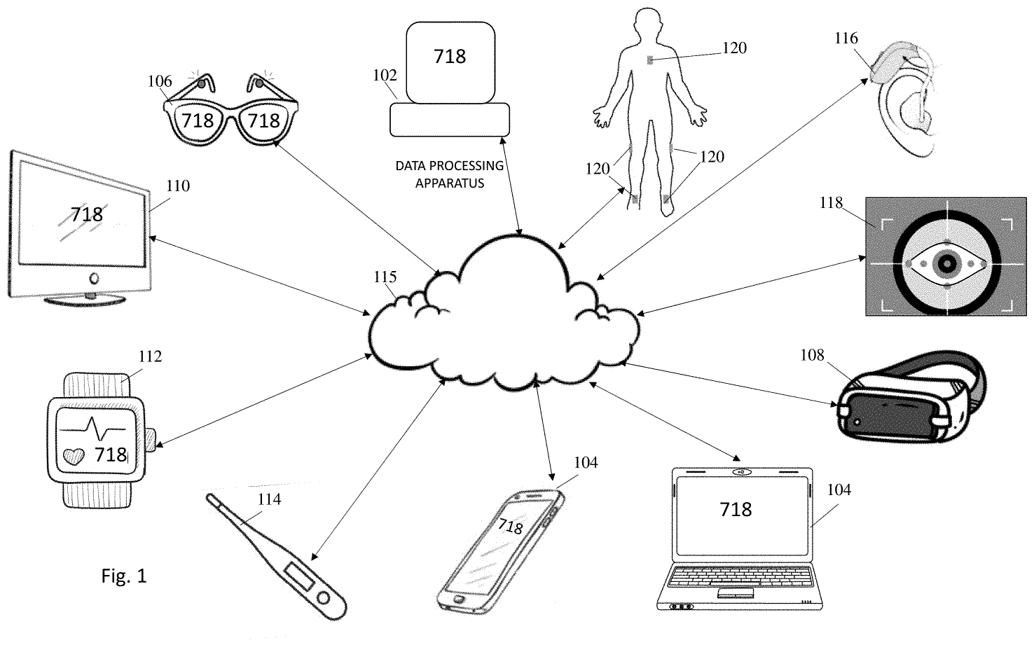

Referring now to the drawings, FIG. 1 is a simple block diagram illustrating a plurality of devices that can be utilized and integrated in accordance with an example implementation of the present disclosure. It is to be recognized that the example devices shown in FIG. 1 are illustrative and other types of computing devices, sensors, measuring devices, I/O devices that are not shown in FIG. 1 are envisioned and can be included herein. In the example shown in FIG. 1, data processing apparatus 102, user computing devices 104, smart glasses 106, virtual reality goggles 108, and display device 110 all can operate to provide input and output to a user, such as visually, audibly, and haptically. The example devices 102, 104, 106, 108, and 110 in FIG. 1 can generate and/or display (or otherwise provide) a user's calculated health score or other representation of the user's state of health. In the example shown in FIG. 1, the example health score value 718 is provided in the respective devices to represent the capability to provide a health score to an individual.

The data processing apparatus 102 can be coupled to one or more user computing devices 104 across communication network 115. User computing devices 104 can include, for example, mobile computing devices such as tablet computing devices, smartphones, personal digital assistants or the like. Further, the plurality of sensing devices can be configured to transmit various health-related information to computing devices, including directly or indirectly to data processing apparatus 102. Moreover, data processing apparatus 102 preferably includes all necessary databases for the present application, However, it is contemplated that data processing apparatus 102 can access any required databases or data source via communication network 115 or any other communication network to which data processing apparatus 102 has access. Data processing apparatus 102 can communicate with such devices comprising databases using any known communication method, including a direct serial, parallel, USB interface, or wirelessly via a local or wide area network.

In addition, the devices in FIG. 1 include sensor and measurement devices, such as smart watch 112, thermometer 114, hearing aid 116, and eye analyzer 118. Each of the devices, or at least components thereof, can interface with one or more computing devices as a function of communication network(s) 115.

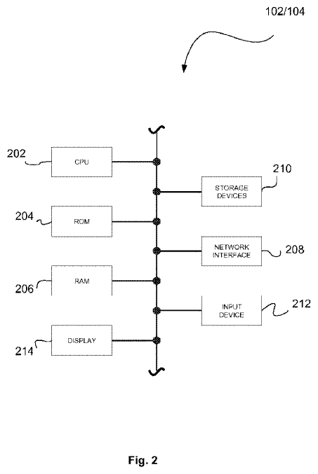

As shown in FIG. 2, the functional elements of each data processing apparatus 102 or user computing device 104, which preferably include one or more central processing units (CPU) 202 used to execute software code in order to control the operation of information processor 202, read only memory (ROM) 204, random access memory (RAM) 206, one or more network interfaces 208 to transmit and receive data to and from other computing devices across a communication network, storage devices 210 such as a hard disk drive, flash memory, CD-ROM or DVD drive for storing program code, databases and application code, one or more input devices 212 such as a keyboard, mouse, track ball and the like, and a display 214.

The various components of data processing apparatus 102 or user computing device 104 need not be physically contained within the same chassis or even located in a single location. For example, as explained above with respect to databases which can reside on storage device 210, storage device 210 may be located at a site which is remote from the remaining elements of data processing apparatus 102 or user computing device 104, and may even be connected to CPU 202 across communication network 115 via network interface 208.

Accordingly, not all elements need be present, for example, storage devices and the capacities of the various elements are arranged to accommodate expected user demand. For example, CPU 202 in user computing device 104 may be of a smaller capacity than CPU 202 as present in data processing apparatus 102. Similarly, it is likely that data processing apparatus 102 will include storage devices 210 of a much higher capacity than storage devices 210 present in user computing device 104. Of course, one of ordinary skill in the art will understand that the capacities of the functional elements can be adjusted as needed.

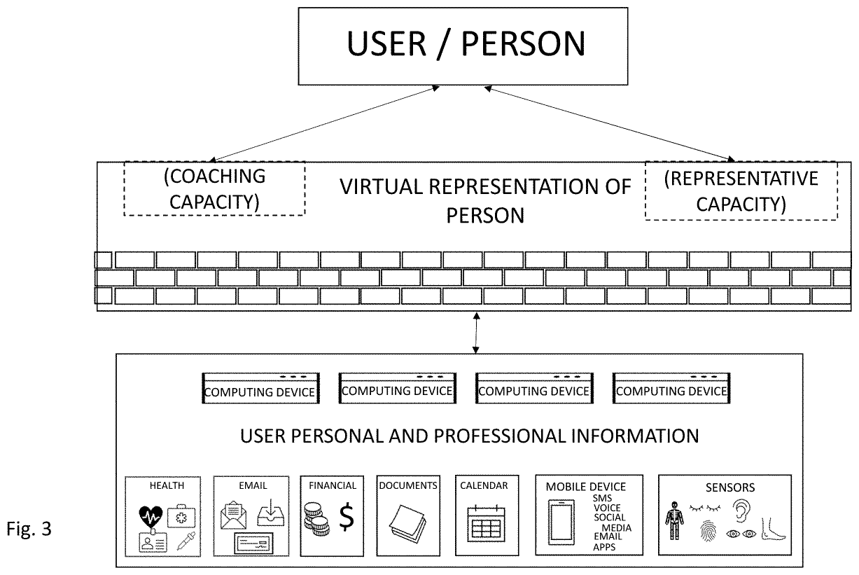

FIG. 3 is a block diagram illustrating a user or person, a virtual representation of a person, respective data sources, and interaction between a user or a person and a virtual representation of a person, in accordance with one or more implementations of the present disclosure. As illustrated in FIG. 3, and as described herein, the virtual representation of a person (e.g., the user) can be provided to the user or person in a coaching capacity or representative capacity depending, for example, on a particular context. The context can be defined in various ways, such as in response to communications sent or received by the user, particular types of information that are being sensed or measured at a given time, or other types of information that are accessible in accordance with the teachings herein. Furthermore, the virtual representation of the person is illustrated to operate as a form of firewall between the user/person and the respective data computing devices, sources of data, and information itself.

For example, and as illustrated in FIG. 3, a plurality of computing devices are shown that can be configured to store and/or access personal and professional information of the user. For example, a user's health information, email, financial information, professional and personal documents, calendar information, information generated and/or accessed by a mobile computing device, such as SMS text, voice information, social media information, email, mobile app data, web sites, or virtually any information associated with a mobile computing device, as well as information that can be sensed and/or measured by one or more components, such as shown and described herein and with reference to FIG. 1.

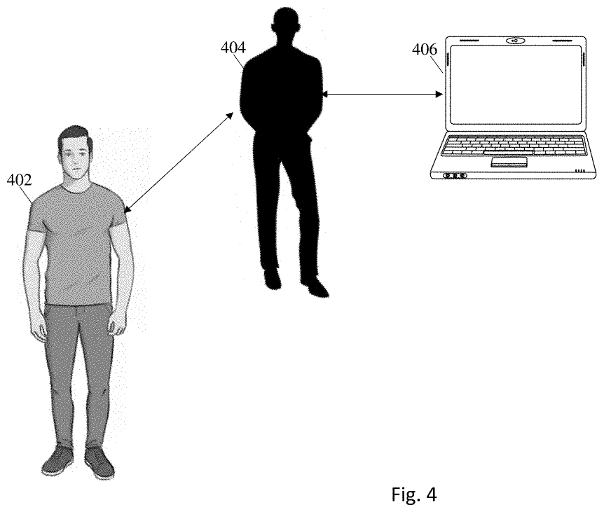

FIG. 4 is a diagram illustrating an individual user (a male 402), a respective virtual representation 404 of the individual user 402, and a respective computing device 406 associated therewith. Although the computing device in FIG. 4 appears as a laptop personal computer, it is to be appreciated that any of the respective devices shown and described herein that can provide a virtual representation of the user (e.g., smartphone 104, virtual reality goggles 108, smart glasses 106, and/or the like) can operate as device 410.

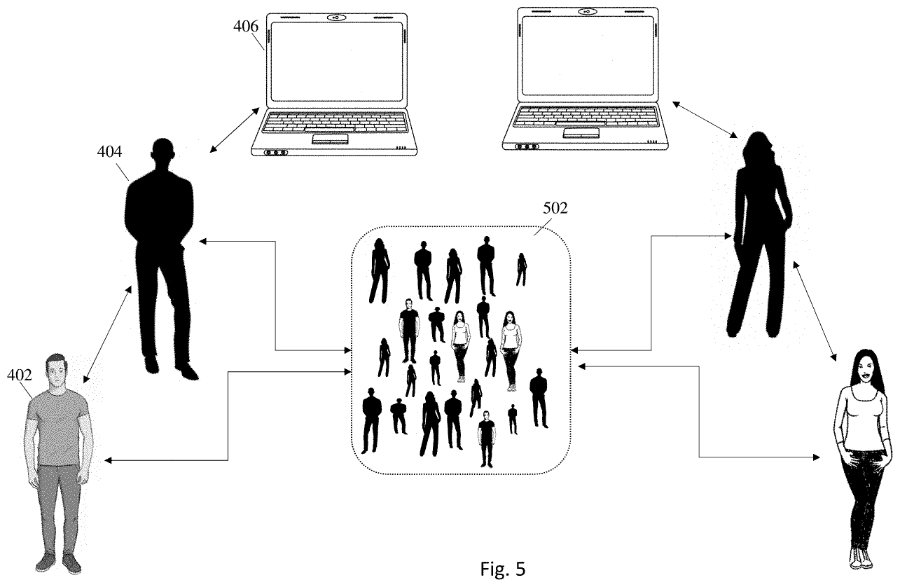

FIG. 5 is a diagram illustrating two respective virtual representations of individual users interfacing in connection with a network 502 of a plurality of other virtual representations of people and individual users. The network 502 illustrated in FIG. 5 shows a plurality of virtual representations of people, as well as physical people. This represents that physical people can interface with virtual representations of people, virtual representations of people can interface with other virtual representations of people, and physical people can interface with each other. Moreover, information that can be provided and/or received by virtual representations of people, such as health data points, documents, or the like.

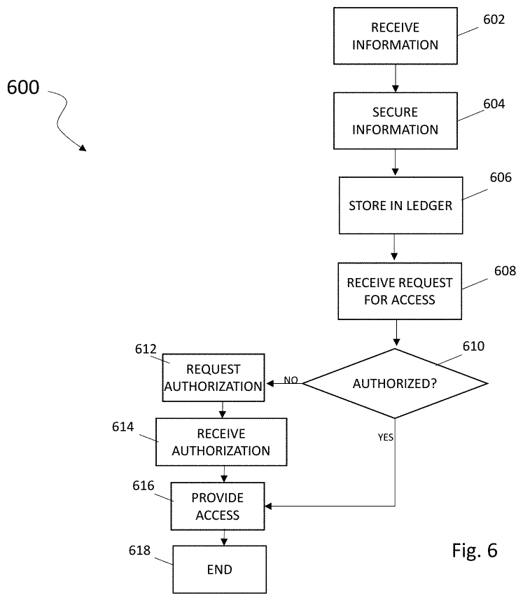

FIG. 6 is a flow chart illustrating example steps 600 that are associated with processing in accordance with an example implementation of the present disclosure. It should be appreciated that several of the logical operations described herein are implemented (1) as a sequence of computer implemented acts or program modules running on a communication device and/or (2) as interconnected machine logic circuits or circuit modules within a communication device. The implementation is a matter of choice dependent on the requirements of the device (e.g., size, energy, consumption, performance, etc.). Accordingly, the logical operations described herein are referred to variously as operations, structural devices, acts, or modules. Several of these operations, structural devices, acts and modules can be implemented in software, in firmware, in special purpose digital logic, and any combination thereof. It should also be appreciated that more or fewer operations can be performed than shown in the figures and described herein. These operations can also be performed in a different order than those described herein.

In the example steps shown in FIG. 6, the process begins at step 602 and at least one computing device receives, from each of a respective data collection sources, information associated with a person. At step 604, the at least one computing device secures at least some of the received information at least as a function of at least one token to provide secured information. Thereafter, at step 606, the secured information is stored on a distributed ledger system. A request to access the secured information is received by the at least one computing device from at least one other computing device (step 608). Thereafter, a determination is made whether at least one permission provided by the person gives authorization to the at least one other computing device to access the secured information (step 610). If the determination in step 610 is that the at least one other computing device does not have authorization to access the secured information, the process branches to step 612 and the at least one computing device provides to the person, via a virtual reality and/or augmented reality (“VR/AR”) environment, a virtual representation of the person requesting authorization for the at least one other computing device to access the secured information. At step 614, the virtual representation of the person receives via the VR/AR environment from the person, the authorization and, thereafter, at step 616, the at least one computing device automatically provides, in response to receiving the authorization, the at least one other computing device with access to the secured information. Alternatively, if the determination in step 610 is that the at least one other computing device has authorization to access the secured information, then the process branches to step 616 and the at least one computing device automatically provides the at least one other computing device with access to the secured information. Thereafter, at step 618, the process ends.

Accordingly, the present application engages and empowers users for sustained behavioral change that that supports long-term health improvements. Automatically generated indications are provided that are associated with a user's well-being and health, substantially in real time. Furthermore, personal goals are defined to improve users' health, and information is automatically generated and provided that enables long-term health improvements and healthy lifestyles. The present application includes modules that interface with a multitude of IOT devices and computing devices, that support tracking, verification, and aggregation of information from disparate sources for modeling and provisioning into a single value. Using artificial intelligence and machine-learning, users' health is strengthened via an interactive computing platform that motivates users to engage in health lifestyles to improve or maintain overall health.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed:

1. A computer-implemented method for securely collecting, generating, and provisioning information, the method comprising:
receiving, by at least one computing device from each of a respective data collection sources, information associated with a person;
securing, by the at least one computing device, at least some of the received information at least as a function of at least one token to provide secured information;
storing, on a distributed ledger system, the secured information;
receiving, by the at least one computing device from at least one other computing device, a request to access the secured information;
accessing, by the at least one computing device, at least one permission provided by the person to determine whether the at least one other computing device has authorization to access the secured information;
where the at least one other computing device does not have authorization to access the secured information:
providing to the person, by the at least one computing device via a virtual reality and/or augmented reality (“VR/AR”) environment, a virtual representation of the person requesting authorization for the at least one other computing device to access the secured information;
receiving, by the virtual representation of the person via the VR/AR environment from the person, the authorization; and
automatically providing, by the at least one computing device in response to receiving the authorization, the at least one other computing device with access to the secured information; and
where the at least one other computing device has authorization to access the secured information:
automatically providing, by the at least one computing device, the at least one other computing device with access to the secured information.

2. The computer-implemented method of claim 1, wherein automatically providing the at least one other computing device with access to the secured information further comprises:
accessing, by the at least one computing device, the at least one token; and
providing, to the at least one other computing device, the at least one token.

3. The computer-implemented method of claim 1, further comprising:
providing, by the at least one computing device via a VR/AR environment, the virtual representation of the person, wherein the request to access the at least some of the secured information is received by the virtual representation of the person.

4. The computer-implemented method of claim 3, wherein the request to access the at least some of the secured information is received by the virtual representation of the person from a virtual representation of another person.

5. The computer-implemented method of claim 1, wherein the at least some of the secured information was previously received from a plurality of respective sources, and further wherein automatically providing the at least one other computing device with access to the secured information further comprises:
accessing, by the at least one computing device, the information previously received from the plurality of respective sources;
aggregating, by the at least one computing device, the information collected from each of a plurality of respective sources; and
securing, by the at least one computing device, the aggregated information as a function of the at least one token.

6. The computer-implemented method of claim 1, further comprising:
using interactions between the person and virtual representation of the person via the VR/AR environment over time, by the at least one computing device, for machine learning and artificial intelligence.

7. The computer-implemented method of claim 6, wherein the secured information includes information associated with the person's health, and further comprising:
recognizing, by the at least one computing device via the machine learning and artificial intelligence, at least one health condition associated with the user.

8. The computer-implemented method of claim 6, wherein the secured information includes information associated with the person's health, and further comprising:
monitoring, by the at least one computing device via the machine learning and artificial intelligence, aspects of the person's health.

9. The computer-implemented method of claim 8, wherein the aspects of the person's health include at least one of blood pressure, skin acidity, body temperature, heartrate, pupil movement, pupil shape, eyelid movement, iris information, sleep information, and stress.

10. The computer-implemented method of claim 1, wherein the authorization received by the virtual representation of the person via the VR/AR environment is in spoken form from the person, and further comprising:
converting, by the at least one computing device using voice recognition technology, the spoken form of authorization to a digital form.

11. A computer-implemented system for securely collecting, generating, and provisioning information, the system comprising:
at least one computing device accessing instructions stored on non-processor readable media that, when executed by the at least one computing device, cause the at least one computing device to:
receive, from each of a respective data collection sources, information associated with a person;
secure at least some of the received information at least as a function of at least one token to provide secured information;
store, on a distributed ledger system, the secured information;
receive, from at least one other computing device, a request to access the secured information;
access at least one permission provided by the person to determine whether the at least one other computing device has authorization to access the secured information;
where the at least one other computing device does not have authorization to access the secured information:
provide to the person, via a virtual reality and/or augmented reality (“VR/AR”) environment, a virtual representation of the person requesting authorization for the at least one other computing device to access the secured information;
receive, by the virtual representation of the person via the VR/AR environment from the person, the authorization; and
automatically provide, in response to receiving the authorization, the at least one other computing device with access to the secured information; and
where the at least one other computing device has authorization to access the secured information:
automatically provide the at least one other computing device with access to the secured information.

12. The computer-implemented system of claim 11, wherein the at least one computing device is further configured by executing instructions stored on non-processor readable media to automatically provide the at least one other computing device with access to the secured information by:
accessing the at least one token; and
providing, to the at least one other computing device, the at least one token.

13. The computer-implemented system of claim 11, wherein the request to access the at least some of the secured information is received by the virtual representation of the person.

14. The computer-implemented system of claim 13, wherein the request to access the at least some of the secured information is received from a virtual representation of another person.

15. The computer-implemented system of claim 11, wherein the at least some of the secured information was previously received from a plurality of respective sources, wherein the at least one computing device is further configured by executing instructions stored on non-processor readable media to automatically provide the at least one other computing device with access to the secured information by:
accessing the information previously received from the plurality of respective sources;
aggregating the information collected from each of a plurality of respective sources; and
securing the aggregated information as a function of the at least one token.

16. The computer-implemented system of claim 11, wherein the at least one computing device is further configured by executing instructions stored on non-processor readable media to:
use interactions between the person and virtual representation of the person via the VR/AR environment over time for machine learning and artificial intelligence.

17. The computer-implemented system of claim 16, wherein the secured information includes information associated with the person's health, and further wherein the at least one computing device is configured by executing instructions stored on non-processor readable media to:
recognize, via the machine learning and artificial intelligence, at least one health condition associated with the user.

18. The computer-implemented system of claim 16, wherein the secured information includes information associated with the person's health, and wherein the at least one computing device is further configured by executing instructions stored on non-processor readable media to:
monitor, via the machine learning and artificial intelligence, aspects of the person's health.

19. The computer-implemented system of claim 18, wherein the aspects of the person's health include at least one of blood pressure, skin acidity, body temperature, heartrate, pupil movement, pupil shape, eyelid movement, iris information, sleep information, and stress.

20. The computer-implemented system of claim 11, wherein the authorization received by the virtual representation of the person via the VR/AR environment is in spoken form from the person, and further wherein the at least one computing device is further configured by executing instructions stored on non-processor readable media to:
convert, using voice recognition technology, the spoken form of authorization to a digital form.
